(19) United States
(12) Patent Application Publication
(10) Pub. No.: US 2019/0131000 A1
(43) Pub. Date: May 2, 2019

(54) CLINICAL TRIAL SUPPORT NETWORK DATA SECURITY

(71) Applicant: Icon Clinical Research Limited, Leopardstown (IR)

(72) Inventors: Ronan FOX, Leopardstown (IR); Sean KELLY, Leopardstown (IR); Thomas O'LEARY, Leopardstown (IR)

(73) Assignee: Icon Clinical Research Limited

(21) Appl. No.: 16/041,290

(22) Filed: Jul. 20, 2018

(30) Foreign Application Priority Data
Nov. 1, 2017 (EP) EP17199576.4

(51) Int. Cl. G16H 10/20; H04L 29/06

(52) U.S. Cl. CPC G16H 10/20; H04L 63/20

(57) ABSTRACT

A clinical trial support network has data processors which perform patient data attribute classification during clinical trial setup by identifying attributes which are sensitive, the attributes being instantiated as terms in clinical trial records. For each identified attribute a risk value or weighting associated with the attribute is stored. After this initialization, in real time a potential data posting is received, such as from a patient using an online portal or from a doctor managing a clinical trial group. The data processors perform real time privacy analysis for the potential data posting, by using the risk values of attributes for which there are terms in the potential posting, to determine an overall risk level for the potential data posting. Once the analysis is performed the potential posting is transmitted or posted according to the result: blocked, full publication, or partial publication. The data processors, when executing algorithms to analyse a potential posting, use additional criteria including term frequency in the posting, count of instances of each term in the posting, and a total count of instances of terms which are potentially sensitive.

### CROSS-REFERENCE TO RELATED APPLICATIONS

This patent application claims the benefit under 35 U.S.C. § 119 to European Patent Application No. EP17199576.4, filed on Nov. 1, 2017, the entirety of which is incorporated herein by reference.

### INTRODUCTION

The present disclosure relates to management of clinical trials, and particularly to computer processing of data concerning patients taking part in trials managed by a clinical trial support network.

Clinical trials require gathering of considerable volumes of data from a wide variety of patients, both relating to medical events specific to medication being trialed and of a personal nature concerning the patients. It is important that sensitive data specific to patients be managed very carefully to avoid breaches of security.

US2005/0065824 (Kohan) describes an approach in which a computer application audits data fields to determine those for encoding, and determines parameters for encoding.

US2004/0199781 (Erickson) describes an approach in which comparisons are performed of data from sources and extraction is used to provide k-anonymity values.

U.S. Pat. No. 8,715,180 (Medtronic) describes an approach to data privacy in which data is parsed and stored in first or second storage areas, and there is controlled access to the storage areas.

U.S. Pat. No. 8,606,746 (Oracle) describes a policy hub for maintaining and managing customer privacy information and privacy preferences and a rules database provides privacy rules.

U.S. Pat. No. 8,650,645 (McKesson Financial Holdings) describes an approach in which hashing of files is performed to protect proprietary data.

US2006/0059149 (Dunki) describes use of a productive database in which records are anonymized, the records including non-static data elements generated or processed by programs and static elements which are invariable.

The present disclosure is directed towards providing data privacy or security with less impact on data processing in the clinical trial support network. Another object is to achieve more comprehensive data security.

### ADDITIONAL REFERENCES

1. Term-weighting approaches in automatic text retrieval. Salton, Gerard, and Christopher Buckley. 24, 1988, Vol. Information processing & management. 5.
2. An information-theoretic perspective of tf-idf measures. Aizawa, Akiko. 39, 2003, Vol. Information Processing & Management. 1.
3. International Telegraph and Telephone Consultative Committee (CCITT). Information Technology—Open Systems Interconnection—Systems Management: Alarm Reporting Function. International Telecommunication Union. [Online] 10 Feb. 1992. [Cited: 27 Jun. 2017.] https://www.itu.int/rec/T-REC-X.733/en.

### SUMMARY OF THE INVENTION

The present embodiments describe a method performed by a clinical trial network comprising digital data processers, user interfaces, and databases, wherein the method comprises steps performed by at least one digital data processor including performing patient data attribute classification during clinical trial setup by identifying attributes which are sensitive, said attributes being instantiated as unstructured terms in clinical trial records. The network may provide a sensitivity risk weighting associated with each of a plurality of attributes, the risk weighting representing a risk value for the associated attribute and being in a range from a minimum to a maximum. The network receives a potential data posting, and performs real time privacy analysis of the potential data posting, by identifying terms and posting parameters and using said risk weightings of attributes to determine a posting risk level for the potential data posting. The network may post data according to the privacy analysis of step either as an unaltered version of the original potential posting, or an edited version of the original potential posting. The data processors may automatically execute these steps for each potential data posting.

Some or all of the steps may be performed in response to guided input of data by a patient according to a patient data interface.

The attribute classification step may be performed per clinical trial as part of a clinical trial design phase. The processors may apply a taxonomy of risk weighting values as some or all of the sensitivity risk weightings.

The attribute classification step may be performed by applying a handle which does not contain any sensitive attributes and which have been communicated to patients as meeting data security eligibility criteria.

A database server may persists the received posting and associated data including inputs from contributors related to the received posting, metadata, and also outputs for the received posting.

The parameters may include derived parameters for the potential data posting to determine said posting risk level. A derived parameter may be an unstructured term frequency in the posting, and/or a count of instances of each unstructured term in the posting, and/or a total count of instances of unstructured terms which are potentially sensitive.

The method may include identifying terms of the posting for handle attributes and omitting them from posting risk calculations.

A parameter may be extracted metadata about the potential posting, and/or a set of automatically-extracted keywords, and/or a tag added or selected manually by a patient, and/or a flag to indicate the output of a preliminary automated privacy analysis, and/or structured data resulting from natural language processing and automated semantic keyword extraction.

The real time privacy analysis may be performed by a processor executing an algorithm as follows:

$$risk ( t , d ) \approx {max}_{0 \leq x \leq 1} \sum tfidf ( t , d , D ) = {max}_{0 \leq x \leq 1} {\sum }_{t}^{n} tf ( t , d ) * idf ( t , D )$$

in which the term frequency part of the algorithm is modified to take into account the privacy risk or importance associated with the specific attribute:

$$tf ( t , d ) = {\sum }_{i = 1}^{n} {tf}_{i} * {weight}_{i} / T$$

where tf(t,d) is a modified measure of the term frequency associated with a posting in a document d,
tfᵢ is a count of instances of a term i in the posting,
weightᵢ is the risk or importance of the term I, and
T is the total count off instances of the terms identified as being potentially sensitive occurring in the current document.

The network may comprise at least one data processor configured to analyze a plurality of potential postings or published postings to identify adverse event patterns, and said analysis may include identifying a pattern of unstructured terms included in potential postings of a plurality of patients. The analysis may include using natural language processing (NLP) techniques.

The method may include a further step of initiating a global search of clinical trial databases for both additional indicators of the adverse events and for potential remedies.

The method include performing semantic analysis of keywords based on publicly available dictionaries and thesauri and taxonomy lookups through similarities and stemming methods to initiate a globalized search.

The network may include a least one curator communication device which performs tasks relating to the quality of the data in the network, and also relating to privacy associated with any publicly-generated and available data.

The data processors may perform real time privacy analysis by activating additional processors as required and determined by the volume of postings being submitted, enabled by a micro services architecture utilising self-healing and annealing infrastructures, deploying new instances as detected by management software.

The method may include automatically obfuscating values and terms to avoid excessive feedback to patient devices, for example by replacing a term with a random set of characters or with a pre-configured string which may have been specified as part of the study/patient setup.

The disclosure also describes a clinical trial network comprising digital data processers user interfaces, and databases, wherein at least one digital data processor is configured to perform steps of:
performing patient data attribute classification during clinical trial setup by identifying attributes which are sensitive, said attributes being instantiated as unstructured terms in clinical trial records,
providing a sensitivity risk weighting associated with each of a plurality of attributes, the risk weighting representing a risk value for the associated attribute and being in a range from a minimum to a maximum;
receiving a potential data posting;
performing real time privacy analysis of the potential data posting, by identifying terms and posting parameters and using said risk weightings of attributes to determine a posting risk level for the potential data posting, and
posting data according to the privacy analysis of either as an unaltered version of the original potential posting, or an edited version of the original potential posting, wherein the data processors automatically the privacy analys for each potential data posting.

The clinical trial network data processors may be configured to perform any of the steps defined above in any embodiment.

The disclosure describes a non-transitory computer readable medium comprising software code for performing steps of a method of any embodiment when executed by a digital data processor.

### DETAILED DESCRIPTION OF THE INVENTION

### BRIEF DESCRIPTION OF THE DRAWINGS

The present disclosure will be more clearly understood from the following description of some embodiments thereof, given by way of example only with reference to the accompanying drawings, in which:

FIG. 1 is a diagram showing a clinical trial network of computers, servers and databases of the invention, according to one or more embodiments;

FIG. 2 is a flow diagram illustrating a workflow for setting up clinical trial data structures so that there is anonymization of data of patients both contributing to clinical trials and in receipt of communication from the network, according to one or more embodiments; and

FIG. 3 is a flow diagram showing real time steps in operation of the network to ensure data privacy, according to one or more embodiments.

### DESCRIPTION OF THE EMBODIMENTS

Network

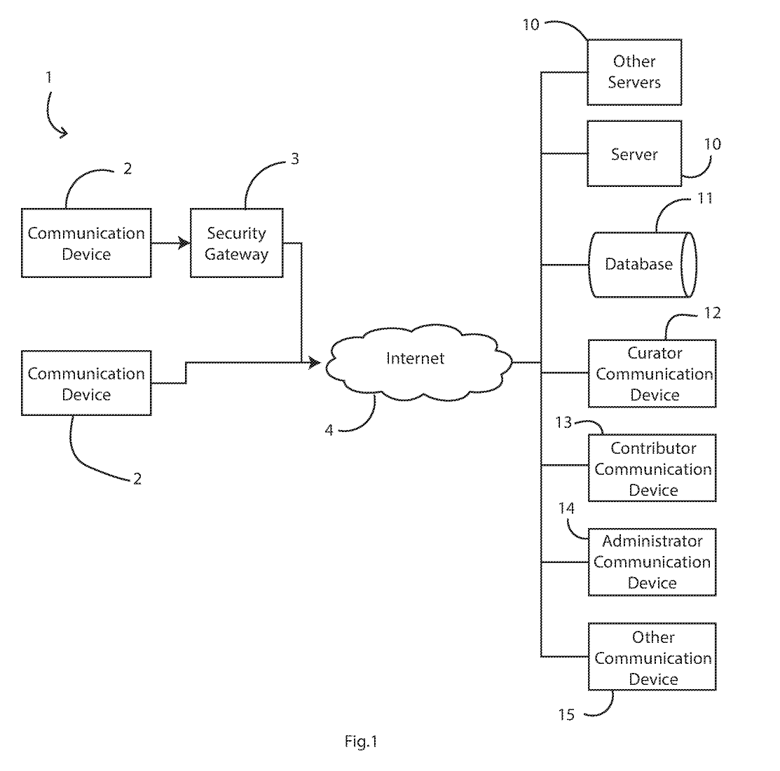

Referring to FIG. 1 a clinical trial support network 1 comprises communication devices 2 linked by a security gateway 3 to the Internet 4. This in turn links the devices 2 with servers 10, a database 11, a curator communication device 12, a contributor communication device 13, an administrator communication device 14, and with other communication devices 15. Each device and server comprises digital data processors and communication interfaces as is well known in the art. The database software comprises a relational database management system which supports a schema required to hold data relating to patient data postings including, but not limited to:
The posting content.
Identity of the person who posted the message
Time and date of the posting
Destination of the posting, such as the patient's clinical trial group, specific member of the group, or other “parent” content (such as the parent blog post if this was a comment on that original “parent” posting”)
Any extracted metadata about the posting, including automatically extracted keywords, manually added tags, flags to indicate the output of initial automated privacy analysis, and structured data resulting from natural language processing and automated semantic keyword extraction.

The servers are configured in hardware terms according to the specified requirements in the clinical trial network. In one example the servers have a speed in the range of 2 to 3 GHz, have in the range of 4 to 16 cores, and a memory capacity of 12 to 15 GB. However, the parameters may be different, depending on the capacity requirements.

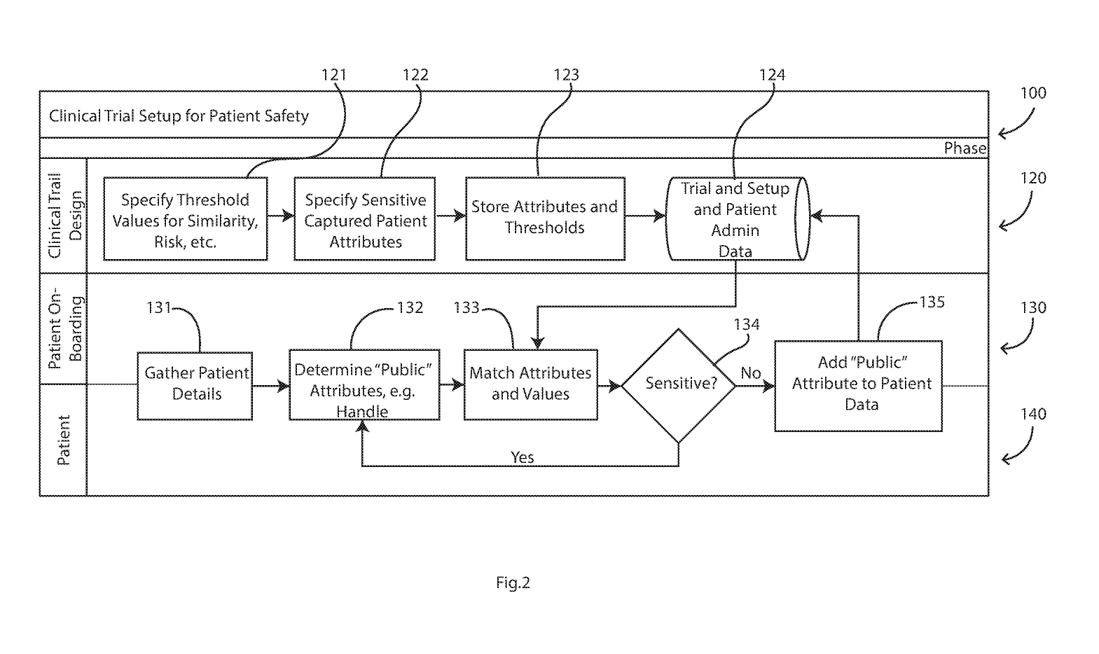

The support network data processors are programmed to perform patient data attribute classification during clinical trial setup (120 in FIG. 2) and patient setup (130, 140 in FIG. 2) by identifying attributes which are sensitive, the attributes being instantiated as terms in clinical trial records. This may be done irregularly, such as one a month or when a re-configuration is desired. For each identified attribute a risk value or weighting associated with the attribute is stored.

The network devices and servers are programmed as follows for automatic processing of potential postings.

The database server 11 is configured to persist the posting and any associated data that may be generated through the administration of the clinical networks, inputs from any contributors, and outputs from any automatic or manual processing performed on the postings and related metadata. The database technology may be instantiated as a document store, and/or as an RDBMS, or as an in-memory data grid spanning clusters of servers to allow for faster throughput and real time processing of postings as they are made to the patient support network. The deployment of the database server may be in a private data centre on in a secured public cloud infrastructure to allow for quick scale up during periods of intense activity in a clinical trial and where the volumes of posts to be analysed may spike during these periods.

The curator communication device 12 provides a means for an “owner” or curator of the network site to perform tasks relating to the quality of the data on site, and also relating to privacy associated with any publicly-generated and available data. This includes providing a means to decide upon and enact processes relating to the publishing or deletion of data as required in the social network, including applying risk weightings to attributes as described in detail below. This device in turn may be part of a peer to peer network of devices which would allow for many people to collaborate in a workflow of authorisations and delegations to allow for multiple opinions and human oversight.

The contributor communication device 13 may be used by professional (often clinical) staff to provide content into the network 1. This content may be of a medical and/or regulatory nature.

The administrator communication device 14 enables a user to administer the network 1. Tasks such as account provisioning and subsequent management are performed by this device.

Other communication devices 15 enable other actors who may interact with the network 1 in roles not described above to for example, link sites, carry out research, etc.

Other servers 10 include servers that may be used in the provisioning of the network 1. Examples of these servers may be firewalls, load balancing servers, and redirection servers which may be required to provide scale, robustness and other non-functional services. As before these servers may be part of a cluster of (virtual) machines which can be deployed as required to satisfy the scale needs of the network.

Clinical Trial Design and Patient On-Boarding

Referring to FIG. 2 a workflow 100 is implemented by the server 10 and the database system 11 of the network 1. There are stages as follows:

120, clinical trial design;

130, patient data on-boarding; and

140, patient data analysis and enrichment during configuration.

The workflow 100 concerns patient setup and includes data relating to a specific patient allowing not just attributes but also specific data values to be marked as sensitive, so that it can be used in the real time processing. There may be a temporal overlap as patients can be on-boarded as entries are being processed, but the aspects illustrated in FIG. 2 all relate to the early clinical trial or study setup and the on-boarding phase of each patient.

In the stage 120 a step 121 specifies threshold values for similarity and risk. Examples are shown in the table below with attributes, example values, and risk/weightings.

In step 122 sensitive patient attributes are specified. The threshold values of step 121 and the attributes are both stored in step 123 into a database 124 of trial set-up and patient administration data.

In more detail, during setup the trial designer decides upon a set of attributes which are considered as sensitive. An attribute is a type of term to be used in a patient record, such as Name or Address or Age, or any term which may be received in a potential posting. Each attribute is given a risk weighting which indicates the risk associated with protecting the privacy of the patient. An example of this attribute selection and weighting is as follows:

| Attribute | Example Value | Risk Weighting |
|---|---|---|
| Patient Name | John Doe | .5 |
| Patient Age | 37 | .03 |
| Patient Street and Number | 41 Appleton Way | .4 |
| Patient County/City | New York | .01 |
| Patient Spouse Name | Mary Doe | .05 |
| Clinical Site Attending | 42^{nd} Street | .01 |

During patient on-boarding for a trial, the attributes for patient details are gathered in step 131, and in step 132, public attributes are determined according to a decision step 134. In step 133 the attributes are matched to values in data retrieved from the database 124. This is fed into the decision step 134 for automatic updating of public attributes. Finally, in step 135 public attributes are added to the patient data of the database 124.

It will be appreciated from FIG. 2 that there is flexibility to allow manual specification of those attributes gathered about the patient in the current clinical trial which are deemed as being sensitive. This provides a taxonomy of search terms that will drive elements of the patient safety maximisation algorithms of the network. The weighting values are between 0 and 1.

Because all attributes are not equally risky for patient data privacy, the weighting between (0,1) achieves excellent flexibility.

A set of “standard” terms is used to give a measure of the sentiment associated with specific postings.

As accounts are being set up by patients or on their behalf a “handle” is specified which will not contain any of the sensitive attributes identified in the setup phase. This handle is used by the patient when engaging in the online patient support network within that specific clinical trial. The “handle” is chosen by the patient but must meet eligibility criteria to ensure that there is no connection to sensitive attributes.

The workflow of FIG. 2 can be enacted as part of the trial setup by study staff or it could form part of a self-service model where the patient executes some of the setup on his/her own behalf.

In the decision step “Sensitive?” 134 a form of NLP using an Apache Lucene index is executed. The specific Lucene search is configurable by use of a FuzzyQuery and can use weightings and other parameters to adjust the type of search done in the query using edit distances, as one example.

As part of the “Sensitive?” 134 step this type of query will remove any commonly used words, combinations of words and word terms which might be specified by the patient or study team during public persona setup in the clinical trial setup phase (or patient recruitment phase). Also, the “Sensitive?” step 134 can also be used to ensure that anonymity of the participants (patients) is maintained with no sensitive data relating to patient identity being published.

Given that the identity of the patient who is posting a contribution to the support network is not known from the decision made when choosing the handle, the remaining task is to ensure that the specific content itself does not expose significant personal information to others on the trial.

Real Time Analysis

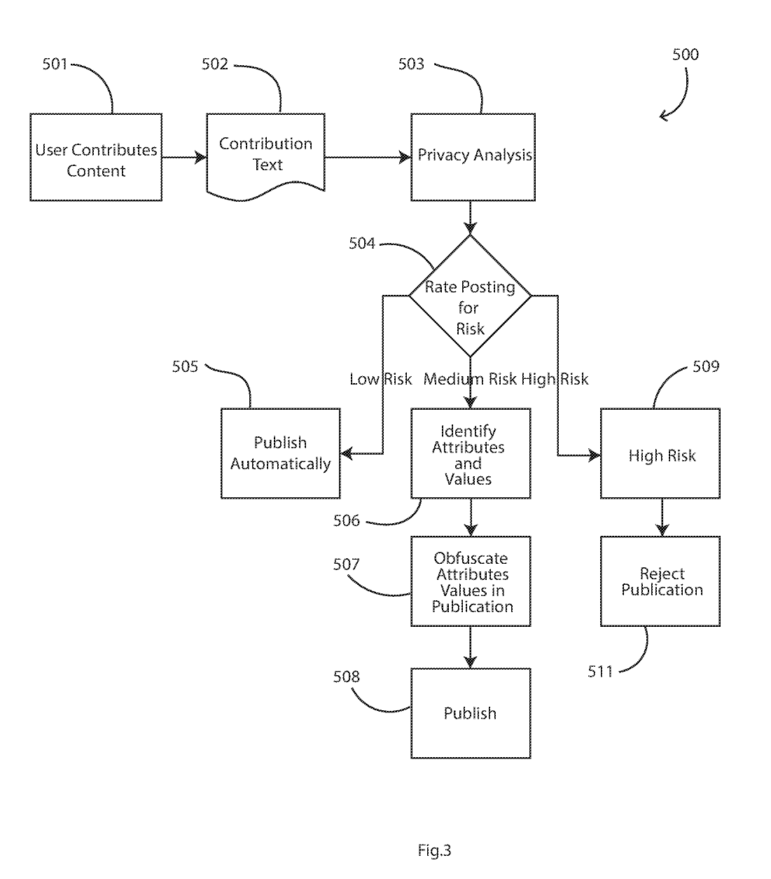

FIG. 3 shows a real-time process 500 that is performed automatically upon calculation of the risk associated with a submitted contribution or “potential posting” from a patient. Specific steps can be customized by use of configurable workflow tools. Advantageously, the network 1 has a customizable filtering mechanism to rate the risk associated with a proposed posting within a clinical trial network, despite the fact that the potential posting includes unstructured data (free form text).

After this initialization, in real time (501 and 502 in FIG. 3) a potential data posting is received, such as from a patient using an online portal or from a doctor managing a clinical trial group. Any of the servers 10 to 15 or any device 2 may provide such a contribution.

The data processors perform real time privacy analysis in step 503 for the potential posting, by using the risk values of attributes for which there are terms in the potential posting, to determine an overall risk level for the potential data posting. This analysis of a posting is done when the server receives the posting content, upon which the workflow in FIG. 3 begins. The processors may be spun up as required and determined by the scale needs of the network and the volume of postings being submitted. This would be enabled through a micro services architecture which will utilize self-healing and annealing infrastructures, deploying new instances as detected by management software (where service drop offline or where the incoming traffic is too much for the existing number of services deployed).

The data in the posting is unstructured, even though it may be provided via a guided patient interface form. There may be fields in a posting form to allow the patient to enter other data such as tags, or a “protect me” flag which might indicate to the curator that the patient is not sure whether they should do this posting, and that the curator should look at it prior to publication even if it passes through the automatic analysis. The data processor recognizes such tags and filters them out. The tags may not need to be processed as part of step 503. The fact that the patient activates a tag indicating a patient-perceived risk is recorded as part of an audit process. In some embodiments activation of such a tag by the patient causes the privacy analysis step 503 to be performed differently by marking the posting as Medium Risk and moving straight to the “Rate Posting for Risk” step (504) where the curator will have the opportunity to review the content as obfuscated by the system prior to publication.

The database server digital processors execute an algorithm for measuring the risk associated with these terms, as follows:

$$risk ( t , d ) \approx {max}_{0 \leq x \leq 1} \sum tfidf ( t , d , D ) = {max}_{0 \leq x \leq 1} {\sum }_{t}^{n} tf ( t , d ) * idf ( t , D )$$

The term frequency part of the algorithm is modified to take into account the privacy risk or importance associated with the specific attribute:

$$tf ( t , d ) = {\sum }_{i = 1}^{n} {tf}_{i} * {weight}_{i} / T$$

where tf(t,d) is a modified measure of the term frequency associated with a posting in a document d,
tfᵢ is a count of instances of a term i in the posting,
weightᵢ is the risk or importance of the term I, and
T is the total count off instances of the terms identified as being potentially sensitive occurring in the current document.

As noted above a “term” is an instance of an “attribute”.

Inverse document frequency is a measure of how unique the term is across all other postings published to date, and thus how much information is exposed by use of the attribute in a specific posting. It is given as:

$$idf ( t , D ) = log \frac{N}{1 + \vert \{ d ∈ D , t ∈ d \} \vert}$$

where N is the total number of published postings
and |{d∈D, t∈d}| is the number of postings where the term d appears

Then, the maximum operator is applied to capture situations where the idf will identify a high weighting and push the idf and thus the risk above 1.

The data processors, when executing algorithms to analyse a potential posting, use additional criteria including, but not limited to:
term frequency in the posting; and/or
count of instances of each term in the posting; and/or
a total count of instances of terms which are potentially sensitive.

Other parameters include the risk or importance associated with the term such that if it was released, it would have a measurable risk of revealing sensitive patient data.

The process may be performed in an offline batch mode. In this example, it might for efficiency run when there are batch sizes of 20 or 30 messages, and release together.

The step 503 results in a step 504 of posting a risk value. Referring also to the ranges in the table below there is:
505, automatic publication if risk is low
506, 507, 508, for medium risk attribute identification, obfuscating values followed by publication (manual intervention may also feature here depending on the setup of the trial);
509, 511, determining high risk and rejecting publication.

Table of Step 504 Posting Risk Ranges

| Risk Range | Meaning | Action |
|---|---|---|
| 0 ≤ x ≤ 0.4 | Low Risk | Publish Automatically |
| 0.4 ≤ x ≤ 0.7 | Medium Risk | Send for Review prior to Publishing |
| 0.7 ≤ x ≤ 1 | High Risk | Do not Publish |

The step of obfuscating values and terms is very advantageous because it means that there is an efficient flow of postings which are safe, avoiding excessive feedback to patient devices. The posting may be an unaltered version of the original posting, or an edited version of the posting to remove private data.

This step is performed as follows:
The identified instance of the sensitive data (for example, the patient name) is either replaced with a random set of characters to prevent any possible linkage to the patient name.
The identified instance could also be replaced with a pre-configured string which may have been specified as part of the study/patient setup. At that point the curator may be given the opportunity to specify fixed replacement values for each sensitive attribute found.

It will be appreciated that, because a risk is determined during setup for each potentially sensitive attribute, real time dynamic decision making is performed as set out in FIG. 3 to ensure privacy. There is therefore very little impact on performance of the clinical trial network.

Once the analysis is performed the potential posting is transmitted or posted according to the result: blocked, full publication, or partial/obfuscated publication.

This network could also identify adverse side effects that a trial is having on patients by identifying conditions using the above taxonomy and calculating a distribution across the patient set. For example, a patient may declare that he/she is experiencing a side effect of a rash while accepting the therapy associated with the trial. A server can use Natural Language Processing (NLP) techniques to identify this side effect through multiple postings from multiple patients either automatically, or through the intervention of a human curator. These NLP techniques are well established in the literature and extend to semantic analysis of keywords based on either publicly available dictionaries and thesauri through to more limited analysis carried out in the form of taxonomy lookups through similarities, stemming, and other well-established methods. This would then initiate that globalized search. From this the trial may also discover the remedies patients are using to treat such rashes which may improve the patient experience and thus the likelihood of them staying on the study until completion. Once a condition is identified further symptoms can be added and applied to historical data to further and quickly ascertain the impact of such side effects supporting immediate action.

The network may identify and filter comments directed at other contributors which could be addressed by extending the taxonomy of keywords to identify such sensitive concepts as “placebo” and “rash”. In a social networking site or a blog site where patients can comment on articles or other comments it would be important to discover and potential curate such entries as might indicate to a patient that they may be on one arm or another within a study. A patient could discover from comments or other postings that they might be taking a placebo and thus upon that realisation leave the trial before completion.

The network can also protect trial-sensitive information from making its way into the public domain, enforcing trial protocol on such matters.

The network could be used to enable inter-site as well as inter-patient social networks where the patent can protect sites from exposing patient or other sensitive data.

Advantages of the Disclosed Embodiments

The present disclosure incorporates structured data fields which are anonymized by allowing their identification and configuration and extending that usage to identifying the specific values within those fields to determine if there is disclosure of patient-sensitive data with unstructured information released by the patient into a social network environment linked with the clinical trial support network 1. It does not need to use bucketing and is focused on the risk associated with unstructured data (free form text) generated by the patient.

The present disclosure does not need to use a configuration or set of preferences per patient which are used to identify identifiable information. It is capable of very efficiently and effectively processing unstructured patient communications and with a set of patient identifiable data elements protect patient data in an online social network for example.

The present disclosure provides a mechanism to protect unstructured data from revealing any sensitive information from a patient, primarily by executing the risk algorithm which forces the data through a review and approval process, but does not use hashing to do so as part of the workflow. Also, the technology of the present disclosure relies on real time ingestion and anonymization of patient data without necessarily having a historical data set with which to work. Rather, it uses profiles and risk assessment to identify potential data elements that could be used to identify a patient at risk and to then redact that information from a real-time interaction such as through a clinical trial support network.

### Alternative Embodiments

The algorithms described for automatically determining a risk value for a posting use the parameters of:
a modified measure of term frequency in a posting,
count of instances of the term in a posting;
a weight reflecting risk or importance of the term;
a total count of instances of an attribute in a posting.

A different algorithm may use different parameters as chosen for particular clinical sites. This allows excellent flexibility.

Also, some of these parameters may be omitted, such as for example the total count parameter.

The present disclosure is not limited to the embodiments described but may be varied in construction and detail.

1. A method performed by a clinical trial network comprising digital data processers user interfaces, and databases, wherein the method comprises steps performed by at least one digital data processor including:
(a) performing patient data attribute classification during clinical trial setup by identifying attributes which are sensitive, said attributes being instantiated as unstructured terms in clinical trial records,
(b) providing a sensitivity risk weighting associated with each of a plurality of attributes, the risk weighting representing a risk value for the associated attribute and being in a range from a minimum to a maximum;
(c) receiving a data posting comprising a potential data posting;
(d) performing real time privacy analysis of the data posting, by identifying terms and posting parameters and using said risk weightings of attributes to determine a posting risk level for the data posting, and
(e) posting data according to the privacy analysis either as an unaltered version of the original posting, or an edited version of the original posting, wherein the data processors automatically perform the privacy analysis and post data according to the privacy analysis for each data posting.

2. A method as claimed in claim 1, wherein some or all of the steps (a) and (b) are performed in response to guided input of data by a patient according to a patient data interface.

3. A method as claimed in claim 1, wherein performing patient data attribute classification includes performing the patient data attribute classification per clinical trial as part of a clinical trial design phase (120).

4. A method as claimed in claim 3, wherein providing a sensitivity risk weighting includes applying a taxonomy of risk weighting values.

5. A method as claimed in claim 1, wherein performing patient data attribute classification includes applying a handle which does not contain any sensitive attributes and which have been communicated to patients as meeting data security eligibility criteria.

6. A method as claimed in claim 1, wherein a database server (11) persists the received posting and associated data including inputs from contributors related to the received posting, and metadata, and also outputs for the received posting arising from step (d).

7. A method as claimed in claim 1, wherein the parameters in performing the real time privacy analysis include derived parameters for the data posting to determine said posting risk level.

8. A method as claimed in claim 7, wherein a derived parameter is an unstructured term frequency in the posting, a count of instances of each unstructured term in the posting, or a total count of instances of unstructured terms which are potentially sensitive.

9. A method as claimed in claim 1, wherein performing real time privacy analysis includes identifying terms of the posting for handle attributes and omitting them from posting risk calculations.

10. A method as claimed in claim 1, wherein for performing the real time privacy analysis, a parameter is extracted metadata about the posting, a set of keywords, a tag, a flag, structured data, or a combination thereof.

11. A method as claimed in claim 1, wherein the network comprises at least one data processor configured to analyse a plurality of postings or published postings to identify adverse event patterns.

12. A method as claimed in claim 11, wherein said analysis of a plurality of postings includes identifying a pattern of unstructured terms included in postings of a plurality of patients.

13. A method as claimed in claim 11, wherein said analysis of a plurality of postings includes using natural language processing NLP techniques.

14. A method as claimed in claim 1, comprising the further step of initiating a global search of clinical trial databases for both additional indicators of the adverse events and for remedies.

15. A method as claimed in claim 1, wherein the network includes a least one curator communication device which performs tasks relating to the quality of the data in the network, and also relating to privacy associated with any publicly-generated and available data.

16. A method as claimed in claim 1, wherein the data processors perform real time privacy analysis by activating additional processors as required and determined by the volume of postings being submitted, enabled by a micro services architecture utilising self-healing and annealing infrastructures, deploying new instances as detected by management software.

17. A method as claimed in claim 1, wherein posting data according to the privacy analysis includes automatically obfuscating values and terms to avoid excessive feedback to patient devices.

18. A method as claimed in claim 17, wherein said step of obfuscating values and terms includes replacing a term with a random set of characters or with a pre-configured string which may have been specified as part of the study/patient setup.

19. A clinical trial network comprising digital data processers user interfaces, and databases, wherein at least one digital data processor is configured to perform steps of:
(a) performing patient data attribute classification during clinical trial setup by identifying attributes which are sensitive, said attributes being instantiated as unstructured terms in clinical trial records,
(b) providing a sensitivity risk weighting associated with each of a plurality of attributes, the risk weighting representing a risk value for the associated attribute and being in a range from a minimum to a maximum;
(c) receiving a data posting comprising a potential data posting;
(d) performing real time privacy analysis of the data posting, by identifying terms and posting parameters and using said risk weightings of attributes to determine a posting risk level for the data posting, and
(e) posting data according to the privacy analysis either as an unaltered version of the original posting, or an edited version of the original posting, wherein the data processors automatically perform the privacy analysis and post data according to the privacy analysis for each data posting.

20. A non-transitory computer readable medium comprising software code for performing steps of a method of any of claim 1 when executed by a digital data processor.
